(19) United States
(12) Patent Application Publication
(10) Pub. No.: US 2010/0041984 A1
(43) Pub. Date: Feb. 18, 2010

(54) Impedance sensing device and catheter system

(76) Inventors: James Edward Shapland, St. Paul, MN (US); Christopher G. Quinn, Minneapolis, MN (US); Rodney W. Salo, Fridley, MN (US)

Correspondence Address: MERCHANT & GOULD PC, P.O. BOX 2903, MINNEAPOLIS, MN, 55402-0903, US

(21) Appl. No.: 12/228,536

(22) Filed: Aug. 12, 2008

(51) Int. Cl. A61B 5/055; A61M 25/10; A61B 5/05

(52) U.S. Cl. 600/424; 604/96.01; 600/547

(57) ABSTRACT

A sensing system including a collection catheter and a sensing device. The sensing system can be used in determining an optimal location of a collection catheter for the removal of a medium from coronary circulation. The sensing system can also be used in determining the optimal time for the collection of blood containing a medium. The sensing system can further be used to maintain a baseline flow rate through at least a portion of the coronary circulation system during a medical procedure.

### FIELD OF THE TECHNOLOGY

The present disclosure relates generally to devices and systems for use in the medical field, and various methods associated with such devices and systems. More particularly, this disclosure relates to devices and systems used in medical procedures involving the removal of contrast media from coronary circulation, and various methods associated therewith.

### BACKGROUND

Coronary circulation is the circulation of blood in the vessels that supply blood to and from the heart muscle (the myocardium). The heart is supplied by the right and left coronary arteries and is drained mainly by veins that empty into the coronary sinus.

Angiography is a medical imaging technique in which an X-ray or fluoroscopic image is taken to visualize the lumen of blood vessels and organs of the body. To assist in the visualization process, a contrast media may be added to the blood.

One of the more common angiography procedures performed is the visualization of the coronary arteries. Typically in this procedure, a catheter is used to administer the contrast media into one of the two major coronary arteries. X-ray images of the contrast media within the blood allow visualization of the size, anatomy, and patency of the arterial vessels.

Contrast media, however, can have significant health risks if permitted to flow systemically to the patient's organs. For example, renal dysfunction or failure may occur from such systemic delivery of contrast media. Such dysfunction or failure is referred to as “contrast-induced nephropathy” or CIN.

Systems and methods have been developed for the removal of the contrast media from the coronary circulation. For example, in some removal methods, a collection catheter is positioned to collect the contrast media as it exits the coronary circulation. In general, conventional systems used to collect and remove contrast media, and the associated conventional methods, can be improved.

### SUMMARY

One aspect of the present disclosure relates to a method for improving the removal of contrast media from coronary circulation by determining an optimal location of a collection catheter. Another aspect of the present disclosure relates to a method for improving the removal of contrast media by determining the optimal time for collection of blood containing contrast media. Yet another aspect of the present disclosure relates to a device and system associated with such methods, which includes the collection catheter and a sensing device.

A variety of examples of desirable product features or methods are set forth in part in the description that follows, and in part will be apparent from the description, or may be learned by practicing various aspects of the disclosure. The aspects of the disclosure may relate to individual features as well as combinations of features, including combinations of features disclosed in separate embodiments. It is to be understood that both the foregoing general description and the following detailed description are explanatory only, and are not restrictive of the claimed invention.

### BRIEF DESCRIPTION OF THE DRAWINGS

FIG. 1 is a diagrammatic representation of a myocardium and coronary circulation system;

FIG. 2 is a schematic representation of the distal end portion of a sensing system embodiment, in accordance with the principles discloses, located within the coronary sinus of the coronary circulation system of FIG. 1;

FIG. 3 is a schematic representation of the proximal end portion of the sensing system of FIG. 2;

FIG. 4 is a graph illustrating Impedance vs. Electrode;

FIG. 5 is a graph illustrating Impedance vs. Detection in Flow over Time, of more than one fluid;

FIG. 6 is a graph illustrating Impedance vs. Time, prior to completion of an angioplasty procedure;

FIG. 7 is another graph illustrating Impedance vs. Time, during an angioplasty procedure;

FIG. 8 is a schematic representation of the distal end portion of another sensing system embodiment, in accordance with the principles disclosed, located within the coronary sinus of the coronary circulation system of FIG. 1;

FIG. 8 is a schematic representation of the distal end portion and the proximal end portion of still another sensing system embodiment, in accordance with the principles disclosed;

FIG. 10 is a schematic representation of the distal end portion of yet another sensing system embodiment, in accordance with the principles disclosed;

FIG. 11 is a schematic representation of the distal end portion of another sensing system embodiment, in accordance with the principles disclosed;

FIG. 12 is a cross-sectional view of a schematic representation of the distal end portion of still another sensing system embodiment, in accordance with the principles disclosed;

FIG. 13 is a schematic representation of the sensing system of FIG. 12 located with the coronary sinus of the coronary circulation system; and

FIG. 14 is a graphical representation of resistivity versus percent of contrast media.

### DETAILED DESCRIPTION

Reference will now be made in detail to exemplary aspects of the present disclosure that are illustrated in the accompanying drawings. Wherever possible, the same reference numbers will be used throughout the drawings to refer to the same or like parts.

Referring to FIG. 1, a diagrammatic representation of a myocardium and coronary circulation system is illustrated. The exact anatomy of the myocardial circulation system varies considerably from person to person. In general, the muscle of the heart is supplied with blood from two main coronary arteries (not shown), which branch into smaller arteries, arterioles and capillaries of the myocardium. The capillaries enable the interchange of oxygen, nutrients, and other substances. Deoxygenated blood within the capillaries then flow into venules, veins, and finally to the coronary sinus.

Material introduced upstream into the coronary arteries will be dispersed among the diverging arterioles and capillaries, which then re-converge into a collection of common venules and vein, and then collected in the coronary sinus. In particular, the myocardium of the heart is fed by the right coronary, left anterior descending and left circumflex arteries. Each of these arteries enters a capillary network that eventually converges into the small and middle cardiac vein (FIG. 1), anterior interventricular vein (not shown), and posterior vein of the left ventricle. These veins are all tributaries of the coronary sinus. Material introduced into any of the aforementioned coronary arteries that travels through the capillary network will enter the coronary sinus; providing an opportunity to collect the material before entering the systemic circulation.

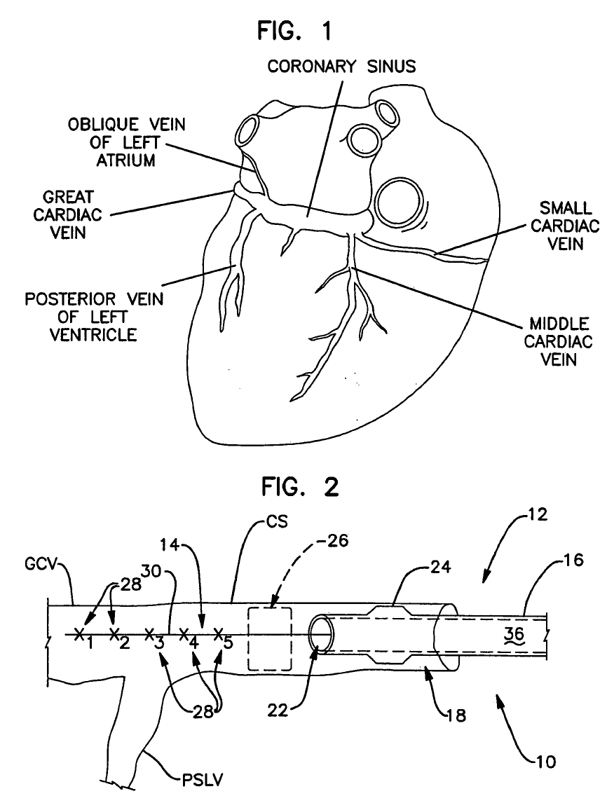

Referring now to FIG. 2, the present disclosure generally relates to a sensing system 10 that senses or detects the electrical properties of a medium flowing through the at least a portion of the coronary circulation system. It is to be understood that “medium” includes, for example, both contrast media and other non-contrast media, such as detection agents (e.g., saline). The detection of the electrical properties of the medium is utilized to optimize the collection location of contrast media introduced into the coronary circulation system, and to optimize the timing for collection of the medium. More specifically, the present disclosure includes a system and method in which the system is used to monitor the impedance of blood in the coronary circulation system to determine the optimal location of a collection catheter, and to determine the optimal timing for removing medium from the myocardium. As such, the present system and method help maximize the removal of a contrast media, for example, during an angiography procedure, and minimize the removal of un-tainted blood.

In another embodiment, the presently disclosed sensing system 10 may be used to determine the anatomy or profile of a vessel surrounding the sensing system. This could further aid in maximizing the removal of a media and minimizing the removal of un-tainted blood by determining the optimal position of a collection catheter within the vessel. In still another embodiment, optical sensing may be used to maximize the removal of a medium. For example, a lack of red blood cells can be optically detected in a procedure during which saline is flushed in the arterial side of the coronary circulation system.

With regards to impedance sensing, blood consists mainly of red blood cells suspended in non-cellular plasma, where the plasma is a solution of proteins and electrolytes, principally sodium and chloride. At low frequencies (i.e. 100 kHz or less) blood can be characterized electrically by its resistivity. The resistivity of a medium determines the current density, which results if a known electric field is impressed on the medium. For example, if charge is injected into blood, the blood presents an impedance to the injected current which is a function of its resistivity. A signal proportional to this impedance may be produced by impressing a known current field in a volume of blood surrounding two points and then measuring the voltage difference between the points. The measured voltage difference varies proportionately with changes in blood impedance, which in turn varies proportionately with changes in blood resistivity. Therefore, changes in blood resistivity due to the addition of contrast media or other solutions with resistivity different from that of undiluted blood will be proportional to changes in blood impedance measured by a pair of electrodes within the blood volume. Resistance is proportional to resistivity, length between the sensing electrodes and inversely proportional to the cross-sectional area of the sensing conduit.

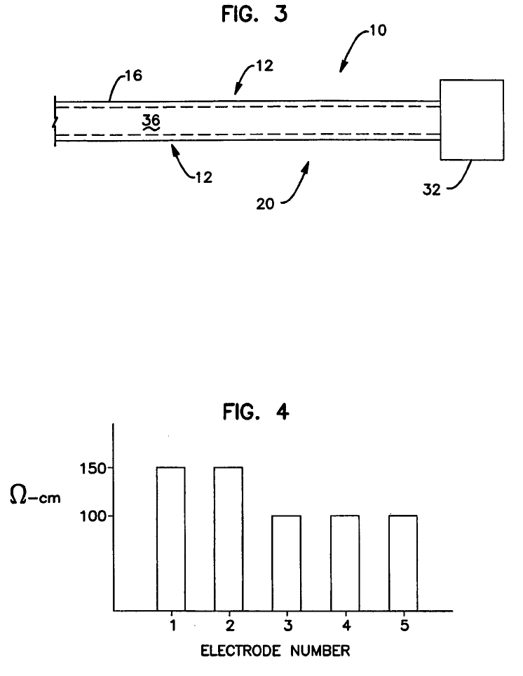

Referring still to FIG. 2, the present sensing system 10 generally includes a collection catheter 12 and an impedance sensing device 14 that function to determine the impedance of a medium as previously described. The collection catheter 12 includes an elongated collection tube 16 having distal end 18 and a proximal end 20 (FIG. 3). An opening 22 is located at the distal end 18. The opening 22 is in fluid communication with a collection lumen 36 defined by the collection tube 16.

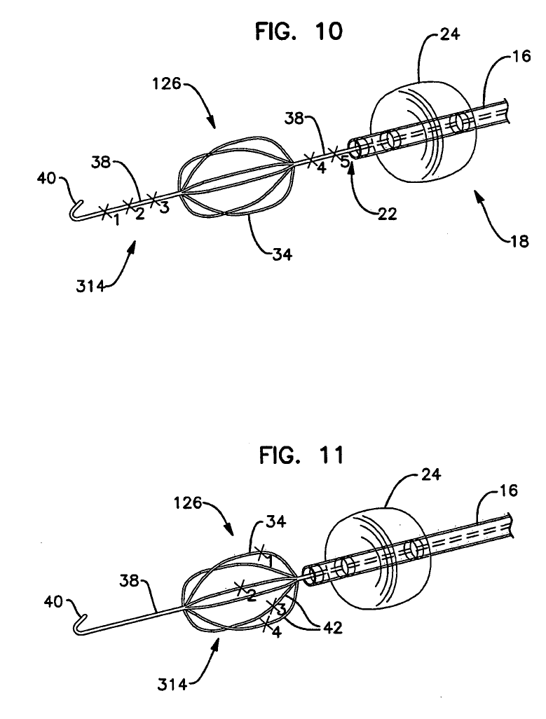

In one embodiment, the collection catheter 12 may include an inflatable balloon 24 or other functional element. The inflatable balloon 24 may be used as a retention balloon and/or an occlusive balloon; although a collection catheter having other retention and/or occlusive devices can also be used. The inflatable balloon 24 is typically located adjacent to the distal end 18 of the collection tube 16 to maintain the opening 22 of the collection tube 16 at a selected location, or to occlude during fluid removal. Further details of example balloons that can be used in the present sensing system are described in U.S. application Ser. No. 11/996,416 and U.S. Publication No. 2008/0108960; which disclosures are incorporated herein by reference in their entirety. The collection catheter 12 can also include a vessel support device 26 that may be deployed to maintain the patency of the coronary sinus CS. In the illustrated embodiment of FIG. 2, the vessel support device 26 is schematically represented and may take any shape known in the art to maintain the patency of a vessel. For example, the vessel support device 26 may be constructed as illustrated in FIG. 10 (see ref. no. 126). Further details of example vessel support devices that can be used in the present sensing system are described in U.S. application Ser. No. 11/557,312 and U.S. Publication No. 2008-0108960; which disclosures are previously incorporated herein by reference in their entirety.

Referring to FIG. 3, the proximal end 20 of the collection catheter 12 located exterior to the patient's body is illustrated. A vacuum 32 is in fluid communication with the collection lumen 36 of the collection tube 16. When actuated, the vacuum draws fluid through the opening 22 (FIG. 2) in the distal end 18 of the collection tube. The fluid is transported through the collection tube 16 to a fluid container exterior of the patient's body for either disposal or for filtering and subsequent return to the patient.

In the illustrated embodiment of FIG. 2, the elongated collection tube 16 is sized to receive the impedance sensing device 14. The sensing device 14 may be received or located within the collection lumen of the tube 16, or may be received or located within a separate lumen. The sensing device is movable relative to the collection tube 16. That is, the sensing device 14 is movable within the receiving lumen (e.g., the collection lumen 36 or a separate lumen) between an extended position (shown in FIG. 2) and a retracted position. In the retracted position, the impedance sensing device 14 is located entirely within the receiving lumen of the collection tube 16.

The impedance sensing device 14 of the present sensing system 10 includes a plurality of electrodes 28 secured to a support or introduction element 30. In the illustrated embodiment, the introduction element 30 is separate from the vessel support device 26, and is separate from the collection tube 16 and collection catheter 12. The electrodes 28 are located at spaced apart distances on the introduction element 30, and in particular, are linearly arranged or aligned in a spaced relationship along a portion of the longitudinal length of the introduction element 30. When the impedance sensing device 14 is in the extended position, the electrodes are external to the collection tube 16 and positioned at spaced distances from the opening 22 of the collection tube.

In the illustrated embodiment, five electrodes 28 are provided on the introduction element 30. The electrodes are equally spaced a distance of about 2 millimeters to 5 millimeters from one another. In other embodiments, the sensing device can includes a greater or lesser number of electrodes spaced farther or closer to one another, or a greater or lesser number of electrodes spaced at varying distances from one another, in accordance with the principles disclosed.

While conventional electrophysiological catheters having electrodes is known, such catheters are typically used to monitor the electrical activations of cardiac tissue during the cardiac cycle. During use, such electrophysiology catheters measure and visualize electrical activity occurring in a patient's heart for electrophysiologic mapping, for example. Additionally, in such mapping procedures, an electric field may be introduced into the heart chamber. The blood volume and the moving heart wall surface modify the applied electric field. Electrodes on the catheter monitor the modifications to the field and a dynamic representation of the location of the interior wall of the heart is developed. In one embodiment, an electrophysiological catheter can be used as the sensing device in the present sensing system 10, in accordance with the principles disclosed.

In one method of use of the presently disclosed sensing system 10, the distal end 18 of the collection catheter 12 is positioned within the coronary sinus CS. During insertion of the collection catheter 12, the sensing device 14 is in the retracted position such that the electrodes 28 and introduction element 30 are located within the lumen of the collection tube 16 so as not to interfere with the insertion of the collection catheter. In the alternative, the collection catheter 12 alone may be positioned within the coronary sinus CS; with the collection catheter 12 so positioned, the sensing device 14 can then be inserted within the lumen of the collection tube 16, and directed toward the opening 22 at the distal end 18.

The sensing device 14 is extended through the opening 22 of the collection catheter 12 a distance from the opening 22. The electrodes 28 of the sensing system 10 can then be used to detect which of the vessels/veins flowing into the coronary sinus CS is/are the main or primary tributary vessels/veins that contain the contrast media. In the illustrated embodiment of FIG. 2, the sensing device 14 has five electrodes 28 that are utilized to determine, for example, whether the great cardiac vein (GCV) or the posterior vein of the left ventricle (PVLV) contains contrast media.

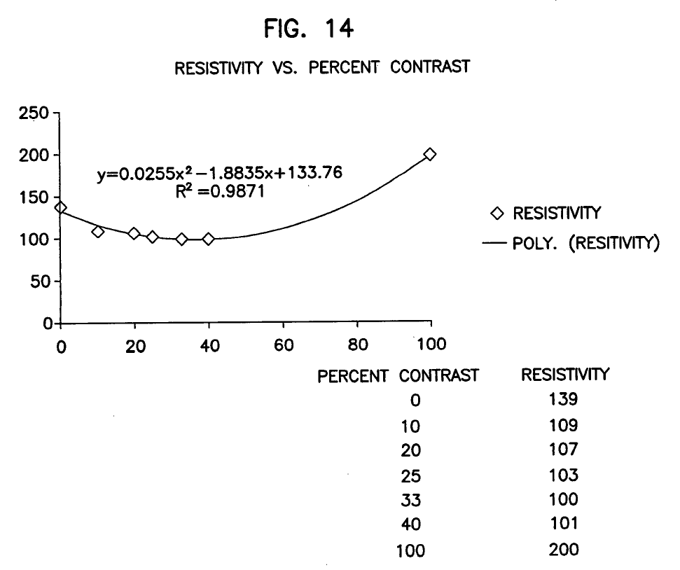

In particular, in determining which vessels/veins carry the contrast media, the impedance measured between each pair of electrodes 28 is monitored as the contrast media clears from the myocardium. Blood typically has a resistivity of about 150 ′Ω-cm. When injected with contrast media, the resistivity decreases, for example, to about 100 ′Ω-cm, which will be detected as a decrease in electrical impedance. Although the resistivity of undiluted contrast medium is about 200 ′Ω-cm, which is greater than that of blood, the resistivity of blood mixed with contrast medium drops to approximately 100 ′Ω-cm, as shown in FIG. 14. If electrodes number 1 and 2 adjacent to the great cardiac vein GCV detect little or no media and electrodes number 3-5 adjacent the posterior vein of the left ventricle PVLV detect media, vessel PVLV is the primary drainage vessel containing the contrast media. Such detection can be shown graphically; see for example FIG. 4 illustrating little or no media detection at electrodes 1 and 2 (i.e., the resistivity measurement is generally that of only blood, ˜150 ′Ω-cm.), and lower resistivity measurements representing media detection at electrodes 3-5.

In this case, the collection catheter 12 can be left in the same location during the angiography procedure, as the collection tube opening 22 is located downstream of the antegrade flow of the primary vessel containing the contrast media. In the alternative, the collection tube opening 22 of the collection catheter 12 can be re-positioned at a closer location to the primary vessel, or can be re-positioned into the primary vessel. Once positioned at the desired location, the balloon 24 can then be inflated to prevent retrograde blood flow (i.e. provide occlusion), and aspiration of the system 10 activated for collection of the contrast media.

In another case, the collection catheter 12 could be designed to be advanced and positioned in the primary vessel containing the contrast media. The collection catheter 12 could include a steerable component or other feature to facilitate cannulation of the primary vessel. The design could also include a balloon that would be inserted into the primary vessel to assist in retaining the catheter in the desired position or provide intermittent vessel occlusion. A further embodiment would incorporate a secondary catheter or extension which would be advanced through the collection catheter 12 into the primary vessel and act as the collection tube 16. This structure could also contain the occlusion/retention balloon.

As can be understood, the sensing system 10 can be utilized to determine the presence or absence of contrast media in vessels/veins other than the great cardiac vein GCV and posterior vein of the left ventricle PVLV illustrated in FIG. 2. For example and referring back to FIG. 1, the sensing system 10 can be used to determine first whether either of the small cardiac vein or middle cardiac vein carries contrast media. If no contrast is detected, the sensing system 10 can be moved or re-positioned adjacent to or at the next vessel/vein that contributes flow to the coronary sinus. The sensing device 14 may be left in the extended position during such re-positioning, or may be retracted and re-extended at each selected position.

In the alternative, the collection tube 16 may remain at a single position within the coronary sinus CS and the sensing device further extended or re-positioned adjacent to or at different vessels/veins. E.g., the sensing device 14 moves from a first extension position to a second extension position relative to the collection catheter 12 to determine by impedance measurements from which particular vein contrast media is flowing. Yet in still other investigatory methods, the sensing device 14 is extended into a vein that flows into the coronary sinus CS, as opposed to being located within the coronary sinus adjacent to the vein. E.g., the collection catheter 12 may be located in the coronary sinus CS while the sensing device 14 is extended into the great cardiac vein (GCV) or even further distally into one of the tributary veins.

In yet another method, the sensing system 10 is used to first detect the primary vessels/veins which will carry the contrast media prior to injection of the contrast media. In particular, a non-toxic agent having a different resistivity from that of blood, referred to herein as a detection agent, is injected into one of the two coronary arteries. The detection agent can have a resistivity that is either higher or lower than that of blood. The detection agent can include, for example, ultrasound beads that contain air pockets and have a higher resistivity than blood, or saline that has a lower resistivity than blood.

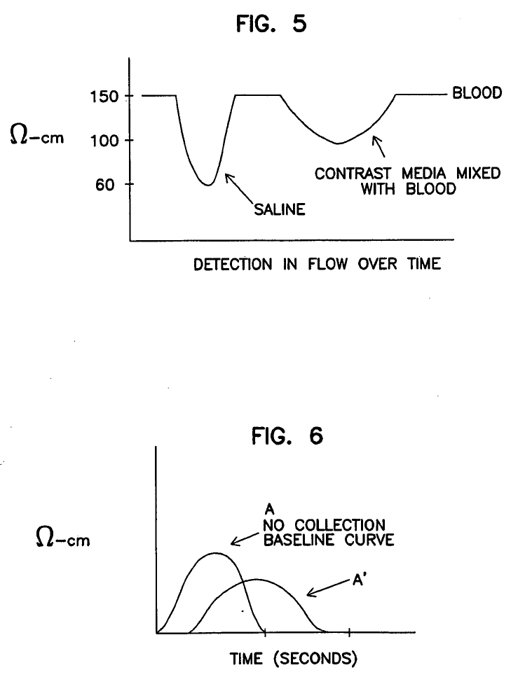

In further example and referring to FIG. 5, saline has a resistivity of about 60 ′Ω-cm, as opposed to blood having a resistivity of about 150 ′Ω-cm and contrast media mixed with blood having a resistivity of about 100 ′Ω-cm. Due to the health risks associated with systemic delivery of contrast media, the non-toxic detection agent, e.g., saline, is first introduced into the coronary circulation system to determine through which vessels/veins the saline, and accordingly the contract media, will flow.

In this detection method, the sensing system is introduced into the coronary sinus CS, as previously described. Saline is injected into one of the two coronary arteries. The impedance measured by pairs of the electrodes 28 (FIG. 2) of the sensing system 10 is monitored as the detection agent is cleared from the myocardium. As previously described, if electrodes number 1 and 2 detect little or no agent and electrodes number 3-5 detect agent, the posterior vein of the left ventricle PVLV is the primary drainage vessel that will carry the contrast media. Alternately, if electrodes 1-2 detect the agent, and there is a higher impedance detection at electrodes 3-5 (higher than saline resistivity of 60 ′Ω-cm due to the dilution of blood from the PVLV vein), then the great cardiac vein GCV is the primary drainage vein. The collection catheter 12 is then advanced to remove blood from vessel GCV, instead of vessel PVLV.

Generally, the above methods of detecting the primary vessels/veins that are carrying contrast media, or that will carry contrast media includes comparing impedance measurements of each electrode pair. An impedance measurement generally equal to that of blood indicates no contrast media/detection agent is present at that corresponding electrode pair. An impedance measurement significantly greater than or significantly less than that of blood indicates contrast media/detection agent is present at the corresponding electrode. What is meant by “significantly” is that the detected impedance change is at least 10% greater than or 10% less than the baseline impedance of blood.

In addition to determining the optimal location of a collection catheter, the sensing system 10 can be used to determine when to activate and deactivate operation of the collection catheter. What is meant by operation of the collection catheter is operation of the vacuum 32 or aspiration device for removing fluid or blood containing contrast media. In this method of use, when a first predetermined level of contrast media is detected by the electrodes 28, operation of the collection catheter 12 is activated, either manually or automatically. At a second predetermined level, operation of the collection catheter 12 is deactivated, either manually or automatically. The second predetermined level can be the same as or different than the first predetermined level. In one example, collection begins at a point in time when an average of resistivity measured at the electrodes drops by ten percent from 150 ′Ω-cm to 135 ′Ω-cm. Collection can be discontinued when an average resistivity measured by the electrodes returns to about 135 ′Ω-cm, or reaches a different predetermined level. Similarly, the activation and deactivation of the collection catheter 12 can be determined by the impedance measured by only one pair or a predetermined number of pairs of electrodes 28. In one such embodiment, activation and deactivation are determined by a minimum impedance value and a maximum impedance value measured by any one pair of electrodes.

Other predetermined levels corresponding to other types of media or detection agents can be utilized. For example, saline is often used to dilute contrast media. The predetermined levels for activation and deactivation can therefore correspond to the detection of saline to sense the arrival of contrast media in the coronary sinus.

In yet another method, the present sensing system 10 can be used to determine the baseline flow rate or transit time of blood through the myocardium and coronary sinus prior to a procedure, and maintain that baseline flow rate during use of the collection catheter. In the alternative, determination of the baseline flow rate can be used to adjust the removal rates from the coronary sinus. For example, it may be desirable to set the removal rate at a level that is equal to or slightly less than the baseline flow rate of blood through the coronary sinus.

In one such method, the sensing system 10 is used to determine optimal inflation of an occlusive balloon. In particular and referring to FIG. 6, a baseline curve (A) is established by injecting a non-toxic detection agent into the coronary artery and observing the time required for the agent to clear the myocardium. This baseline curve A is established when aspiration of the collection catheter is deactivated and when the balloon is deflated. Then another injection of the non-toxic detection agent is introduced into the coronary artery, at which time aspiration of the collection catheter is deactivated but the balloon is inflated. The time required for the agent to again clear the myocardium is observed and a second curve (A′) is generated. The sensing device 14 is utilized in accordance with the principles disclosed in generating curves A and A′.

The second curve A′ is often shifted to the right from that of the baseline curve A when the balloon is inflated (due to the occlusion of the vessel). If the second curve A′ is shifted farther than desired, parameters of operation of the collection catheter can be adjusted to shift the second curve A′ back toward the baseline curve A. For example, the balloon can be partially deflated so as to not be overly occluding in the particular application. In one embodiment, the desired inflation volume of the balloon provides only partial occlusion during use of the collection catheter. Such desired occlusion can be determined by monitoring the sensing system.

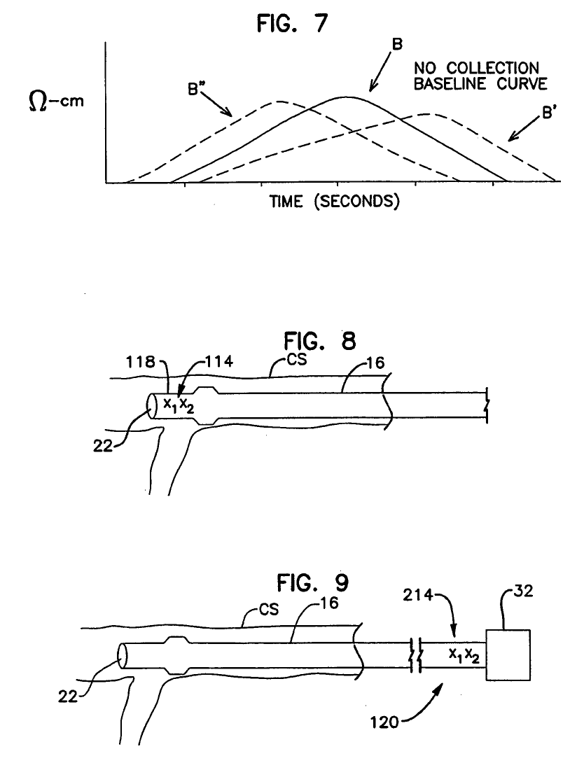

In another method, the sensing system 10 is used to determine an optimal operation of the collection catheter during aspiration. In particular and referring to FIG. 7, a baseline curve (B) is first established by injecting a non-toxic detection agent into the coronary artery and observing the time required for the agent to clear the myocardium. This baseline curve B is established when aspiration of the collection catheter is deactivated; the balloon may or may not be inflated (partially or fully). Then another injection of the non-toxic detection agent is introduced into the coronary artery, at which time of the injection, aspiration of the collection catheter 12 is activated. The time required for the agent to again clear the myocardium is observed and another curve (B′ or B″) is generated. The sensing device 14 is utilized in accordance with the principles disclosed in generating curves B and B′ or B″.

A second curve that is shifted to the right (e.g., curve B′) indicates that flow through the myocardium has been reduced. If desired, the vacuum of the collection catheter can be increased to shift the curve B′ back toward the baseline curve B. A second curve that is shifted to the left (e.g., curve B″) indicates that flow through the myocardium has been increased. To compensate, the vacuum can be reduced to shift the curve B″ back toward the baseline curve B.

In use, both the methods described above with regards to FIGS. 6 and 7 can be used to optimize both the inflation of a balloon and the operation of the collection catheter. In a preferred method, the optimal inflation of the balloon is first determined as described above, then the optimal aspiration/operation of the collection catheter is determined as described above.

In still another method, the sensing system 10 can be used to determine baseline transit time for a medium to flow through the heart to the coronary sinus. For example, the sensing system 10 can be used to detect the arrival of a medium in the myocardium. This transit or delay time information can be used in conjunction with the start time of the injection of the medium, or in and of itself, to further determine when to activate aspiration of the collection catheter. The sensing system 10 can also be used to determine baseline transit time for the medium to clear through the myocardium. For example, the sensing system 10 can be used to detect the clearing of the medium in the coronary sinus. This information can be used in conjunction with the start time and/or end time of the injection of the medium, or in and of itself, to determine when to deactivate aspiration of the collection catheter.

Referring now to FIG. 8, an alternative embodiment of a sensing system including a sensing device 114 with electrodes (e.g., x1, x2) is schematically illustrated. The electrodes of the sensing device 114 are carried on a distal end portion 118 of the collection catheter 12 itself, either internally (within the lumen) or externally of the lumen. In one embodiment, the electrodes of the sensing device 114 can include band electrodes or dot electrodes, for example, that are incorporated into the exterior or interior surface of the collection tube wall at the distal end portion 118. In one embodiment, two electrodes are located at the distal end portion 118 of the collection tube 16. The sensing electrodes are equally spaced a distance of about 1 millimeter to 1 centimeter from one another. In one embodiment, the electrode spacing is between about 2 millimeters and 5 millimeters. In another embodiment, the electrode spacing is less than one-fourth the diameter of the surrounding vessel.

Referring to FIG. 9, in another embodiment, a sensing device 214 can include electrodes (such as band electrodes or dot electrodes) or a flow-through cell, located within the collection tube 16 at a proximal end portion 120 of the catheter. In this embodiment of FIG. 9, the sensing device 214 is external of the patient and monitors the impedance of fluids passing through the collection tube 16 prior to the vacuum 32. In one embodiment, two or more electrodes are located within the collection tube 16. The electrodes are equally spaced a distance of about 2 millimeters to 5 millimeters from one another. In other embodiments, the sensing devices of FIGS. 8 and 9 can includes a greater or lesser number of electrodes spaced farther or closer to one another, or a greater or lesser number of electrodes spaced at varying distances from one another, in accordance with the principles disclosed.

Referring to FIG. 10, yet another embodiment of a sensing system including a sensing device 314 with electrodes is illustrated. The electrodes of the sensing device 314 are carried by a vessel support device 126 that maintains the patency of a vessel during operation of the collection catheter. The vessel support device 126 is typically located upstream from the opening 22 of the collection tube 16 such operation.

The illustrated vessel support device 126 generally includes an expandable basket 34 attached to a wire 38. The wire 38 includes a distal guiding tip 40 that permits the vessel support device 126 to be deployed from the collection tube 16 atraumatically. In one embodiment, the electrodes (e.g., x1, x2, x3) are carried on the wire 38 of the vessel support device 126 between the basket 34 and the guiding tip 40. In another embodiment, the electrodes (e.g., x4, x5) are carried on the wire 38 between the basket 34 and the opening 22 of the collection tube 16. In still another embodiment, electrodes are carried on the wire 38 both forward of and rearward of the expandable basket 34. Referring to FIG. 11, the electrodes (e.g., x1, x2, x3, x4) can instead, or also, be provided on struts 42 of the expandable basket 34. Each of the alternative embodiments in FIGS. 8-11 can be used in the methods described herein and in accordance with the principles of the present disclosure.

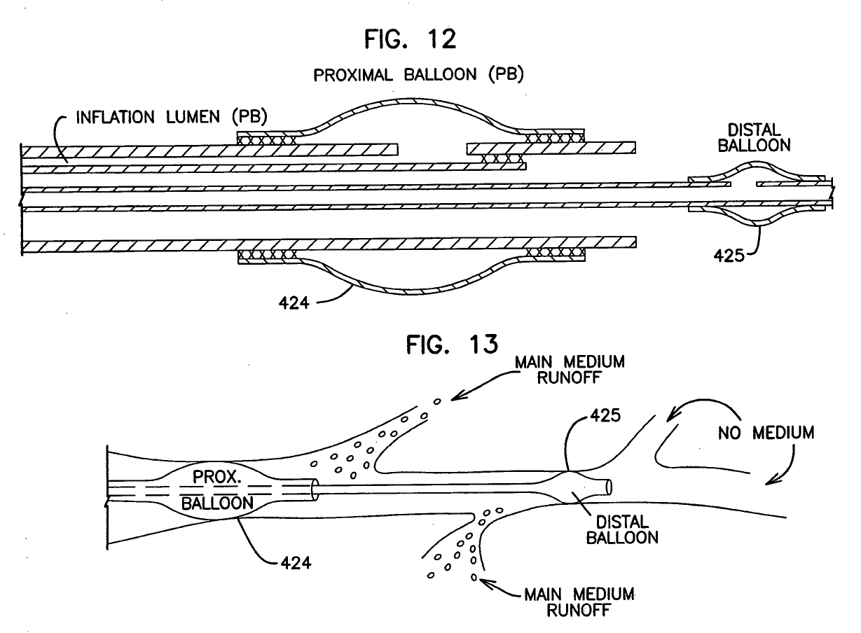

In another embodiment, the sensing device may act as a guide wire and be positioned first within the coronary sinus, with the collection catheter later passed over the sensing device into proper position. Referring to FIGS. 12 and 13, in still another embodiment, high concentration runoff vessels can be isolated by deploying two or more occlusion (partial or full) balloons 424, 425 proximal and distal to the target vessel(s). The balloons 424, 425 can be slidably engaged to allow one balloon (e.g., 425) to move without movement or adjustment of the other balloon (e.g., 424). The two or more balloons can be provided on the same catheter or on different catheters. One of the balloons can also be integral to the sensing catheter and used to detect and determine optimal run-off vessels as previously described.

In addition to use during angiography procedures, the present sensing system 10 can also be used during an angioplasty procedure. In an angioplasty procedure, a narrowed or obstructed blood vessel is mechanically widened to increase flow through the blood vessel. The sensing system 10 aids in determining whether the angioplasty procedure is successful by monitoring the rate at which blood flows through the myocardium.

For example, flow rate through the myocardium is graded against a TIMI flow grading system (having grades of 0 to 3); the lowest grade of “0” indicating complete occlusion of the particular artery and the highest grade of “3” indicating normal flow. Referring back to FIG. 6, the present sensing system 10 can be used to generate the baseline flow curve through the coronary sinus before the procedure, and generate a second flow curve after the procedure. An increase in flow rate indicates that a blockage has been successfully removed.

The above specification provides a complete description of the present invention. Since many embodiments of the invention can be made without departing from the spirit and scope of the invention, certain aspects of the invention reside in the claims hereinafter appended.

What is claimed is:

1. A method of locating a collection catheter within a coronary sinus, the method comprising the steps of:
a) providing a sensing device at a distal end of the collection catheter, the sensing device including at least a first pair of electrodes;
b) positioning a distal end of the collection catheter within the coronary sinus at a first position;
c) monitoring the impedance measured by the first pair of electrodes to detect the presence of a medium flowing into the coronary sinus;
d) determining by the impedance measurements from which particular vein the medium is flowing; and
e) moving the collection catheter to a second position such that an opening at the distal end of the collection catheter is located downstream of the antegrade flow from the particular vein.

2. The method of claim 1, further including extending the sensing device through the opening located at the distal end of the collection catheter.

3. The method of claim 2, further including extending the sensing device from a first extension position to a second extension position relative to the collection catheter to determine by the impedance measurements from which particular vein the medium is flowing.

4. The method of claim 1, further including inflating a balloon when the collection catheter reaches the second position to occlude the coronary sinus during operation of the collection catheter.

5. The method of claim 4, further including removing the medium from the coronary sinus by operation of the collection catheter.

6. The method of claim 5, further including deploying a vessel support device when the collection catheter reaches the second position to maintain the patency of the coronary sinus during operation of the collection catheter.

7. The method of claim 1, wherein the sensing device further includes a second pair of electrodes, and wherein the step of monitoring the impedance includes monitoring the impedance measured by each of the first and second pairs of electrodes, the first pair of electrodes being located farther from the opening of the collection catheter than the second pair of electrodes.

8. The method of claim 7, wherein the step of determining from which particular vein the medium is flowing includes comparing a first impedance measured by the first pair of electrodes with a second impedance measured by the second pair of electrodes, and wherein an impedance measurement generally equal to that of blood indicates no medium is present at that corresponding electrode pair, and wherein an impedance measurement different from that of blood indicates medium is present at that corresponding electrode pair.

9. The method of claim 1, further including providing a vessel support device that maintains the patency of a vessel during removal of the medium, the sensing device being carried by the vessel support device.

10. The method of claim 1, wherein the medium is a contrast media.

11. An impedance sensing system, comprising:
a) an elongated collection tube having a distal opening, the elongated collection tube defining a lumen;
b) a vacuum in fluid communication with the collection tube; and
c) an impedance sensing device sized for receipt within the lumen of the elongated collection tube, the impedance sensing device being movable between an extended position and a retracted position relative to the collection tube, the impedance sensing device including a plurality of electrodes positionable at a spaced distance from the distal opening of the elongated collection tube and external to the collection tube.

12. The system of claim 11, wherein the electrodes of the impedance sensing device are carried by an introduction element inserted within the lumen of the elongated collection tube.

13. The system of claim 12, further including a vessel support device that maintains the patency of a vessel, the vessel support device being separate from the introduction element which carries the electrodes.

14. The system of claim 13, further including an inflatable balloon located adjacent to a distal end of the elongated collection tube.

15. The system of claim 12, wherein the electrodes are aligned along a length of the introduction element.

16. The system of claim 11, further including a vessel support device that maintains the patency of a vessel, wherein the electrodes of the impedance sensing device are carried by the vessel support device.

17. A method of identifying from which particular vein contrast media will flow, prior to injecting contrast media into a coronary artery, the method comprising the steps of:
a) providing a sensing system including a collection catheter and a sensing device;
b) positioning the sensing system such that a distal end of the collection catheter and a first pair of electrodes of the sensing device are located within the coronary sinus;
c) injecting a non-toxic detection agent into a coronary artery;
d) monitoring the impedance measured by the first pair of electrodes to detect the presence of the detection agent flowing into the coronary sinus;
e) determining by the impedance measurements from which particular vein the detection agent is flowing; and
f) adjusting the positioning of the collection catheter such that a distal opening of the collection catheter is located downstream of the antegrade flow from the particular vein.

18. The method of claim 17, wherein the step of providing a sensing system includes providing a sensing device that is carried on a distal end portion of the collection catheter.

19. The method of claim 17, wherein the step of providing a sensing system includes providing a sensing device that is received within a lumen defined by the collection catheter.

20. The method of claim 19, further including extending the sensing device through the distal opening of the collection catheter.

21. The method of claim 20, further including extending the sensing device from a first extension position to a second extension position relative to the collection catheter to determine by the impedance measurements from which particular vein the detection agent is flowing.

22. The method of claim 17, wherein the step of positioning the sensing system includes positioning the sensing system such that the first pair of electrodes and a second a pair of electrodes are located within the coronary sinus, the first pair of electrodes being located farther from the distal opening of the collection catheter than the second pair of electrodes, and wherein the step of monitoring the impedance includes monitoring the impedance measured by the first and second pairs of electrodes.

23. The method of claim 22, wherein the step of determining from which particular vein the detection agent is flowing includes comparing a first impedance measured by the first pair of electrodes with a second impedance measured by the second pair of electrodes, and wherein an impedance measurement generally equal to that of blood indicates no detection agent is present at that corresponding electrode, and wherein an impedance measurement significantly greater than or significantly less than that of blood indicates detection agent is present at that corresponding electrode.

24. The method of claim 17, further including providing a vessel support device that maintains the patency of a vessel, the sensing device being carried by the vessel support device.

25. A method of removing a medium from a coronary sinus, the method comprising the steps of:
a) providing an impedance sensing device including at least one pair of electrodes, the impedance sensing device being located at a distal end of a collection catheter;
b) positioning the distal end of the collection catheter within the coronary sinus;
c) utilizing measured values of impedance from the at least one pair of electrodes to calculate the level of medium in the flow through the coronary sinus;
d) activating operation of the collection catheter to remove the medium from the coronary sinus when the level of medium in the flow is calculated at a first predetermined level.

26. The method of claim 25, further including deactivating operation of the collection catheter when the level of medium in the flow is calculated at a second predetermined level.

27. The method of claim 25, wherein the step of providing the impedance sensing device includes providing a device having a plurality of electrodes, wherein adjacent electrodes define pairs of electrodes.

28. The method of claim 27, wherein the step of utilizing the measure values of impedance includes calculating an average impedance measured by the pairs of electrodes.

29. The method of claim 27, wherein the step of utilizing the measured values of impedance includes:
a) determining a minimum impedance value measured by any one pair of electrodes, and wherein operation of the collection catheter is activated when the minimum impedance value corresponds to the first predetermined level; and
b) determining a maximum impedance value measured by any one pair of electrodes, and wherein operation of the collection catheter is deactivated when the maximum impedance value corresponds to a second predetermined level.

30. The method of claim 25, further including extending the impedance sensing device through an opening located at the distal end of the collection catheter.

31. The method of claim 25, wherein the medium is a contrast media.

32. An impedance sensing system, comprising:
a) an elongated collection tube having a proximal end and a distal end, the elongated collection tube defining a collection lumen in fluid communication with an opening located at the distal end of the collection tube;
b) a sensing device received within the collection lumen of the collection tube, the sensing device being movable between an extended position and a retracted position relative to the collection tube, the sensing device including a plurality of electrodes, each electrode of the plurality of electrodes being aligned with one another and spaced a distance of about 2 millimeters to 5 millimeters from one another.

33. An impedance sensing system, comprising:
a) an elongated collection tube having a proximal end and a distal end, the elongated collection tube defining a collection lumen in fluid communication with an opening located at the distal end of the collection tube;
b) a plurality of electrodes carried within the collection lumen of the collection tube, the electrodes being located outside of the patient's body during operation of the sensing system, the plurality of electrodes including at least two electrodes aligned with one another and spaced a distance of about 2 millimeters to 5 millimeters from one another.
